(19) United States
(12) Patent Application Publication
(10) Pub. No.: US 2020/0383685 A1
(43) Pub. Date: Dec. 10, 2020

(54) CONVERTED ANEURISM CLIP

(71) Applicant: Aesculap AG, Tuttlingen (DE)

(72) Inventor: WOLFGANG SAUTER, Renquishausen (DE)

(21) Appl. No.: 16/095,734

(22) PCT Filed: Apr. 19, 2017
(86) PCT No.: PCT/EP2017/059292

§ 371 Date: Oct. 23, 2018

(30) Foreign Application Priority Data
Apr. 25, 2016 (DE) 10 2016 107 587.6

(51) Int. Cl. A61B 17/122

(52) U.S. Cl. CPC A61B 17/1227

(57) ABSTRACT

A surgical clip includes two clip branches which are spring-elastically joined and pretensioned at proximal ends by a first spring element which can be rotated about a spring pivot point formed by the first spring element. The distal free ends of the clip branches are held in parallel against one another in a resting position of the surgical clip via the closing force of the first spring element. The surgical clip has a conversion device which reduces a ratio of opening force, applied to a force-guiding path for opening the clip branches, to opening angle, from a predetermined opening angle of the distal free ends of the two clip branches. A method for opening the clip includes the step of applying pressure to the clip branches on a force-guiding path formed between crossing portions and the first spring element.

### RELATED APPLICATIONS

This application is the United States entry of International Application No. PCT/EP2017/059292, filed Apr. 19, 2017, which claims the benefit of priority of German Application No. 10 2016 107 587.6, filed Apr. 25, 2016. The contents of International Application No. PCT/EP2017/059292 and German Application No. 10 2016 107 587.6 are incorporated by reference herein in their entireties.

### FIELD

The present invention relates to a surgical clip, especially aneurism clip, comprising two clip branches which are spring-elastically joined and pretensioned at the proximal ends thereof via a first spring element, especially a leg spring, which can be rotated about a spring pivot point formed by the first spring element and which are held with the distal free ends thereof in parallel against one another in a resting position of the surgical clip via the closing force of the first spring element. The invention further relates to a method for opening a surgical clip.

### BACKGROUND

Surgical clips of this type which include two clip branches or jaw part branches, which are joined to each other at the proximal ends thereof via a spring-elastic element and which are held with the distal free ends thereof in parallel against one another in a resting position of the clip by a predetermined closing force are known from the state of the art. It is noted that in the present application the terms “proximal” and “distal” are to be understood with respect to an operating surgeon/physician/user and “proximal” thus denotes the direction toward the user and “distal” denotes the direction away from the user and, resp., toward the patient. The known surgical clips and, resp., aneurism clips make use of a leg spring as spring-elastic element and preferably include crossing portions between the proximal and distal ends thereof at which the clip branches are mutually crossing. Pressure for opening the clip branches is introduced especially to a force-guiding path arranged between the crossing portions and the leg spring.

In surgical clips of this type the closing force thereof is substantial which has to be precisely adjusted and also has to be permanently given when the surgical clip is used as an implant. Such surgical clips are used, to be sure, to pinch off an aneurism, i.e. an arterial dilatation or arterial bulge, which is understood to be a spindle-shaped or bag-shaped, localized and permanent extension of a blood vessel cross-section, and thus to exclude the aneurism from the blood circulation. Surgical clips and, resp., aneurism clips have to be compact and space-saving, as in surgery blood vessels and, resp., aneurisms and the like frequently have to be pinched off in a spatially very constricted environment.

In order to open such surgical clip and, resp., aneurism clip, the ends of the clip branches located proximally from the crossing portion are seized by a tool, for example clip applying forceps and are approximated to each other against the spring force of the leg spring. In so doing, a force or load applied through the tool to the spring and an opening width or an opening angle of the distal ends of the clip branches are proportional relative to each other. In other words, with an increasing opening angle the spring force is continuously increasing. Due to the high closing force of the leg spring required as well as the compact design of the aneurism clip, a working area and, resp., an opening width of the leg spring is limited in the surgical clips and, resp., aneurism clips known from the state of the art. The reachable opening angle and, resp., the reachable opening width is limited, in other words, in the state of the art due to the closing force required and the compact design of the clip required.

### SUMMARY

Said drawbacks of the state of the art are intended to be avoided or at least reduced by the present invention. Especially, a surgical clip or aneurism clip is to be provided which with the previously obtained closing force offers a substantially larger opening width at the distal ends of the clip branches or, resp., jaw part branches without increasing the geometry and, resp., the outer dimensions of the surgical clip.

The invention first relates to a surgical clip, especially aneurism clip, comprising two clip branches/jaw part branches which are spring-elastically joined and pretensioned at their proximal ends via a first spring element, in particular a leg spring, which can be rotated about a spring pivot point formed by the first spring element/about a spring axis of rotation formed by the first spring element and which are held with the distal free ends thereof in parallel against one another in a resting position of the surgical clip by the closing force of the first spring element, wherein the surgical clip has a conversion device which reduces a ratio of opening force applied to a force-guiding path for opening the clip branches to opening angle from a predetermined opening angle of the distal free ends of the two clip branches. The predetermined opening angle preferably is very small and is within a range between 0° and 20°, further preferred between 0° and 10°. Advantageously, the two clip branches include crossing portions between the proximal and distal ends thereof at which the clip branches are mutually crossing, wherein the opening force, especially pressure, for opening the clip branches is applied to the force-guiding path arranged between the crossing portions and the first spring element.

By a surgical clip of this type it is achieved, on the one hand, that in the resting position of the clip the required closing force is preferably exclusively applied via the first spring element which according to the invention is a leg spring, for example, and thus the free distal ends of the clip are held to be in parallel against each other. Hence, the required closing force for closing the clip is provided. According to the invention, said closing force has not or only partly to be overcome for opening the surgical clip, however, for it is achieved by the conversion device of the present invention that from a predetermined opening angle a force to be applied for opening the surgical clip as compared to the closing force of the clip is reduced and/or a force for opening the clip branches applied to the force-guiding path is converted to a larger opening width and, resp., a larger opening angle.

One advantageous example embodiment of the present invention is characterized in that at least one of the clip branches includes an additional pivot point/an additional axis of rotation which is arranged distally from the spring pivot point and proximally from the force-guiding path and about which the at least one clip branch can be rotated while overcoming a spring-elastic pretension of at least a second spring element, especially a leaf spring or a flexible spring.

According to the present invention, it may thus be provided to overcome a spring-elastic pretension of at least a second spring element, which is preferably a leaf spring or a flexible spring, for opening the surgical clip. The second spring element and, resp., the pretension thereof in the closing direction of the clip is designed so that, as compared to the first spring element generating the closing force, lower spring force has to be overcome so that the at least one clip branch of the surgical clip can be opened more easily. In accordance with the invention, said clip branch then rotates during opening the clip while deflecting/bending the second spring element about an additional pivot point/an additional axis of rotation which is arranged between the spring pivot point and the force-guiding path. The additional pivot point thus is closer to the force-guiding path than the spring pivot point of the first spring element. Thus, by such displacement or shifting of the pivot point when opening the at least one clip branch closer toward the force-guiding path, a converted opening width and, resp., a converted opening angle of the at least one clip branch is advantageously obtained.

In other words, deflection of the at least one clip branch at the force-guiding path due to the additional pivot point located more closely to the force-guiding path results in a larger obtainable opening width and, resp., a larger obtainable opening angle of the surgical clip. Thus, according to the invention, with a given closing force a substantially larger opening width of at least one clip branch is obtained at the distal ends of the clip branches and, resp., jaw part branches without increasing the geometry and the outer dimensions of the surgical clip. By providing the second spring element, especially the force to be applied for opening the surgical clip is reduced from the predetermined opening angle provided according to the invention. By providing an additional pivot point of at least one clip branch located more closely to the force-guiding path a converted opening width and, resp., a converted opening angle of the at least one clip branch can be realized.

One advantageous example embodiment is characterized in that each of the two clip branches has an additional pivot point about which the two clip branches can be rotated while overcoming a spring-elastic pretension of second and third spring elements each of which is especially in the form of a leaf spring or a flexible spring, wherein the two additional pivot points are preferably equally spaced distally from the first spring element and proximally from the force-guiding path.

Thus, according to this embodiment, at the one clip branch a first additional pivot point and a second spring element are provided and at the other clip branch a second additional pivot point and a third spring element are provided. According to this embodiment, each of the two clip branches thus necessarily includes the afore-described conversion device. In this way, according to this embodiment both clip branches are deflectable to a larger extent and the opening width to be obtained and, resp., the opening angle to be obtained are thus increased. Advantageously, the two additional pivot points are equally spaced apart distally from the first spring element and proximally from the force-guiding path so that symmetric deflection of the two clip branches can be ensured when the surgical clip is opened. In other words, the distance of the first additional pivot point from the first spring element at the one clip branch is equal to the distance of the second additional pivot point from the first spring element at the other clip branch.

It is of advantage when the surgical clip includes a spring travel limiting unit, especially a stop, which limits deflection and/or an opening angle of the two clip branches about the spring pivot point formed by the first spring element. Especially, by such spring travel limiting unit/by such stop the predetermined opening angle and, resp., the predetermined opening width according to the invention of the distal ends of the two clip branches is defined.

According to the invention, it may be provided that the closing force of the first spring element initially has to be partly overcome and rotation of the two clip branches takes place about the spring pivot point formed by the first spring element. Said partial overcoming of the closing force of the first spring element takes place up to a predetermined point or stop and, resp., opening angle defined by the spring travel limiting unit. The point or stop is defined, according to the invention, such that the force required for rotation of the clip branches about the spring pivot point does not become excessively high. The stop limits the rotation of the clip branches about the spring pivot point so that, when further pressure is applied, no further deflection of the distal ends of the clip branches about the spring pivot point is possible any more.

The conversion device according to the invention is thus characterized by a spring travel limiting unit for limiting deflection about the spring pivot point formed by the first spring element and by a pivot point changing unit which, after limiting the deflection of the clip branches about the spring pivot point, performs a change of the pivot point of the clip branches more closely toward the force-guiding path and enables rotation about the new pivot point by bending the second and/or third spring element(s).

It is of particular advantage when upon application of force to the force-guiding path at first a rotation of the clip branches about the spring pivot point takes place and only subsequently a rotation of the clip branches about the at least one/the two additional pivot points takes place while the second spring element/the second and third spring elements is/are deflected. This helps to achieve that in a closing position of the surgical clip the closing force is applied in any case by the first spring element only. Hence the second spring element/the second and third spring elements is/are prevented from influencing/impairing/reducing the closing force of the first spring element.

One advantageous example embodiment of the present invention is characterized in that the two clip branches include a spring portion and a connecting portion at each of their crossing portions, the spring portion of the one clip branch including the second spring element and the spring portion of the other clip branch including the third spring element, and each of the spring portions being connected to the first spring element, wherein the connecting portions of the two clip branches are rotatably joined to each other such that the additional pivot points of the two clip branches are in the form of a joint additional pivot point and each of the clip branches is rotatable about the joint additional pivot point via deflection and/or bending and/or overcoming of the spring-elastic pretension of the second and third spring elements.

In accordance with said example embodiment, it is thus provided that each of the two clip branches splits into two portions toward the proximal end. One portion or, resp., the spring portion is connected to the first spring element and, resp., the leg spring and includes the second spring element and, resp., the third spring element. The spring portions are provided outside of the two clip branches so that pressure is introduced for opening the surgical clip at the spring portions. The other portions and, resp., the connecting portions of the two clip branches are connected or coupled to each other and form the joint additional pivot point. The pivot point is located, especially when viewed in the longitudinal direction, on a central axis of the surgical clip and, resp., in a plane spanned by the contact surfaces of the two clip branches in the closed state of the clip. The connecting portions thus are provided at the inside of the two clip branches. It is understood that, according to the core idea of the present invention, the additional pivot point is in turn arranged proximally from the force-guiding path and distally from the first spring element and, resp., the leg spring. The clip branches according to this example embodiment on the one hand are connected to each other via the first spring element and, resp., the leg spring, on the other hand the clip branches are also connected to each other at the additional joint pivot point. Thus, an additional joint pivot point is provided about which each of the two clip branches is rotatable. According to this embodiment, the force to be applied is reduced especially by the spring portions of the two clip branches comprising the second and, resp., third spring elements and the converted opening width is obtained especially by the joint additional pivot point which is formed at the connecting portions. When a pressure for opening the clip branches is introduced to the spring portions, deflection/bending of the second and third spring elements provided at the respective spring portions and an accompanying rotation of the two clip branches, especially of the connecting portions and the distal ends, about the joint additional pivot point take place.

It is advantageous when the connecting portions of the two clip branches are rotatably connected to each other via a pin joint, wherein the connecting portion of one clip branch includes a pin projecting in the direction of the other clip branch and the pin engages in a recess of the other clip branch and, in this way, a hinge-type rotatable connection is formed between the connecting portions of the two clip branches, with the additional pivot point and, resp., the additional pivot axis being formed by the pin.

According to the invention, the joint additional pivot point of the two clip branches thus is formed especially by a pin joint. As the connecting portion of one clip branch includes a projecting pin engaging in a recess in the connecting portion of the second clip branch, the additional joint pivot point is easily formed. However, according to the invention, the pin may also be provided to be designed as a separate component which in turn engages in recesses provided in the respective connecting portions of the two clip branches.

One advantageous example embodiment is characterized in that the pin has a rotational degree of freedom and a translational degree of freedom and the spring travel limiting unit is formed by limitation of the translational motion of the pin.

Due to the translational degree of freedom of the pin, especially in the direction of force transmission, thus during force transmission at first the two clip branches are deflected about the first spring element and, resp., the leg spring. Subsequently, the translational motion is limited by the spring travel limiting unit according to the invention and, resp., the stop. Thus, the deflection of the two clip branches about the spring pivot point of the leg spring is limited. When further force is applied, finally deflection/bending of the second/third spring elements, especially of the provided leaf springs or flexible springs, and involved rotation of the clip branches about their joint additional pivot point will take place. In this way, it is realized that when applying force to the clip branches, initially rotation of the branches about the spring pivot point takes place and only later or subsequently rotation about the additional joint pivot point will take place. Thus, it is changed over to the joint additional pivot point and the second and third spring elements are deflected before the force for overcoming the closing force of the first spring element becomes excessively high. Changing over to the joint additional pivot point entails force increasing to a smaller extent as well as a converted opening width. The fact that at first merely a translational motion of the pin and no rotational motion, i.e. no deflection of the second and third spring elements, either, takes place, helps to ensure that in a closing position of the clip the closing force is applied by the first spring element only and thus no negative impact is given by the second/third spring elements with respect to the closing force.

It is expedient in this context when the recess of the other clip branch is in the form of a slotted hole, wherein the pin is arranged, in a resting position of the surgical clip, on a rounded side of the slotted hole, is translationally movable in the slotted hole by the introduction of pressure for opening the surgical clip and the spring travel limiting unit is formed by applying the pin to the other rounded side of the slotted hole.

By the design of the recess as a slotted hole the translational motion of the pin is enabled in a simple manner. The stop for limiting the translational movement is performed, according to this embodiment, by the pin contacting a rounded side of the slotted hole. The contact of the pin with the rounded side moreover enables the subsequent rotational movement of the pin and thus the further opening of the clip branches. Thus, configuration of the recess as a slotted hole easily provides both a translational degree of freedom and a rotational degree of freedom of a pin accommodated therein.

Advantageously, according to the invention, when introducing pressure for opening the surgical clip thus at first mainly a translational motion of the pin takes place in the provided recess and the two clip branches are deflectable about the spring pivot point formed by the first spring element, wherein, after limiting the translational motion by the spring travel limiting unit, the rotational motion of the two clip branches about the additional pivot point formed by the pin takes place while the second and third spring elements are simultaneously deflected/bent.

In accordance with another aspect, according to the invention it is further expedient when the second and third spring elements are formed integrally and/or from one single material with the two clip branches and are preferably formed by a suitable structural dimensioning of the spring portions of the two clip branches.

The integral and/or single-material design of the second and third spring elements with the spring portions help to achieve that no subsequent assembly of the second and third spring elements is required. The integral and, resp., single-material design of the second and third spring elements with the spring portion is especially achieved by the fact that at the crossing portions where each of the clip branches splits into the spring portion and the connecting portion, the spring portions are very thin, i.e. having a small material depth and, resp., having a smaller cross-section, and thus the spring elements are configured especially in the way of a leaf spring or flexible spring, and the spring portions are formed toward the force-guiding path up to the first spring element in turn somewhat thicker having a larger material depth and, resp., having a larger cross-section, so that from a predetermined force the thinner portions of the spring portions may be deflected/bent.

Moreover, the invention relates to a method for opening a surgical clip, especially an aneurism clip, especially a clip as afore-described, wherein the surgical clip includes two clip branches which at their proximal ends are spring-elastically joined and pretensioned via a first spring element, in particular a leg spring, which can be rotated about a spring pivot point formed by the first spring element, which are held with the distal free ends thereof in parallel against one another in a resting position of the surgical clip via the closing force of the first spring element and which have crossing portions between the proximal and distal ends thereof at which the clip branches are mutually crossing, characterized by the following steps of: applying a pressure to the two clip branches on a force-guiding path formed between the crossing portions and the first spring element and, in this way, deflecting of the two clip branches about the spring pivot point; limiting the deflection about the spring pivot point by a spring travel limiting unit, especially a stop; further applying pressure to the two clip branches and thus deflecting at least one clip branch about an additionally provided pivot point which is arranged distally from the spring pivot point and proximally from the force-guiding path while bending/overcoming a spring-elastic pretension at least of a second spring element, especially a leaf spring or a flexible spring.

In other words, the invention relates to a converted aneurism clip. By the use of at least two springs (one leg spring and one flexible spring/leaf spring) the working areas of the springs can be segmented or separated so that an opening width of the aneurism clip can be increased without exceeding the limits of the leg spring or, resp., primary spring. By changing the pivot point in the clip branch system and, resp., jaw part branch system, with a smaller angular movement of the leg spring a converted rotation of the clip branches can be obtained while the flexible spring/leaf spring is slightly bent. According to the invention, thus a larger opening width and, resp., a larger working area of the clip branches is obtained with a high closing force.

### BRIEF DESCRIPTION OF THE DRAWING FIGURES

Hereinafter the invention shall be illustrated in detail by way of figures, wherein:

FIG. 1 shows a longitudinal view of an embodiment of a surgical clip according to the invention in a closing position of the clip branches;

FIG. 2 shows a longitudinal view of the clip of FIG. 1 in a slightly opened state briefly before a change of the pivot point;

FIG. 3 shows a longitudinal view of the clip of FIG. 1 and FIG. 2 in a widely opened state after change of the pivot point;

FIG. 4 shows a schematic view illustrating the effects of the change of pivot point according to the invention on the opening width of the two clip branches;

FIG. 5 shows a diagram which illustrates a relation according to the invention between a branch opening angle at the distal ends of the clip branches and a resulting force to be applied; and

FIG. 6 shows a diagram which illustrates a relation according to the invention between a deflection angle at a force-guiding path and a resulting branch opening angle at the distal ends of the clip branches.

The figures are merely schematic and serve exclusively for the comprehension of the invention. Like elements are provided with like reference numerals. The features of the individual example embodiments can be interchanged.

### DETAILED DESCRIPTION

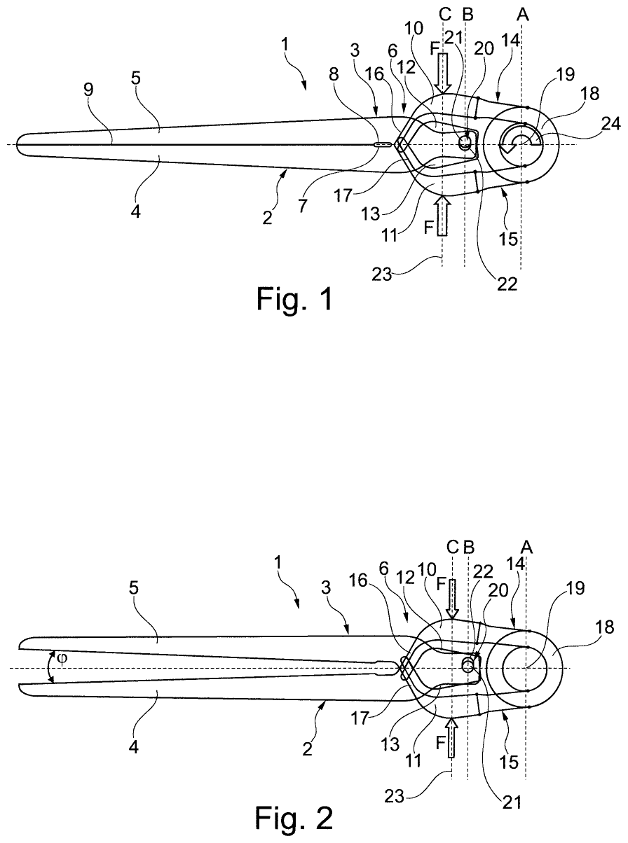

In FIG. 1 an aneurism clip 1 is shown. The aneurism clip 1 comprises a first clip branch 2 and a second clip branch 3. Each of the clip branches 2, 3 includes free distal ends 4, 5 which in the closing position of the aneurism clip 1 shown in FIG. 1 are held in parallel against each other and apply an appropriately high closing force for pinching off an aneurism. The two clip branches 2, 3 are mutually crossing at a crossing area 6, wherein the clip branches 2, 3 are adjacent to each other in parallel in the closing position until just before the crossing area 6. Just before the crossing area 6 the clip branches 2, 3 include recesses 7, 8 which limit a contact surface 9 of the clip branches 2, 3 in the closing position toward the proximal direction.

At the crossing area 6 the two clip branches 2, 3 split into outside portions or spring portions 10, 11 and inside portions or connecting portions 12, 13. The spring portion 10 and the connecting portion 12 together form the proximal end 14 of the first clip branch 2. The spring portion 11 and the connecting portion 13 together form the proximal end 15 of the second clip branch 3. The outside portions or spring portions 10, 11 are configured to be very thin in the crossing area 6 and hence form leaf springs 16, 17.

After the crossing area 6 toward the proximal direction the outside spring portions 10, 11 are thicker, i.e. are formed to have a larger cross-section and are finally connected to a leg spring 18. The leg spring 18 forms a leg spring pivot point 19 about which the two clip branches 2, 3 can be basically rotated. In the closing position of the aneurism clip 1 the closing force is applied to the distal ends 4, 5 of the clip branches 2, 3 by the leg spring 18. The connecting portions 12, 13 of the two clip branches 2, 3 are configured to be substantially thicker in the crossing area 6, i.e. with a larger cross-section, than the spring portions 10, 11.

The connecting portions 12, 13 are joined via a pin joint 20. The pin joint 20 is configured so that the connecting portion 12 of the clip branch 2 includes a pin 21 which engages in a slotted hole recess 22 provided in the connecting portion 13 of the clip branch 3. The pin 21 is arranged in the resting position or closing position of the aneurism clip 1 at an upper end in FIG. 1 of the slotted hole recess 22. A force F for opening the aneurism clip is applied to the spring portions 10, 11 of the two clip branches 2, 3 at a force-guiding path 23. The force-guiding path 23 is predetermined by the maximum extension of the aneurism clip 1 in the height direction. When respective forces F are applied against each other to the spring portions 10, 11 in the configuration of FIG. 1, at first this entails a rotation of the clip branches 2, 3 about the leg spring pivot point 19 with the direction of rotation 24 stated in FIG. 1. The pin joint 20 is arranged proximally from the force-guiding path 23 and distally from the leg spring 18, i.e. between the force-guiding path 23 and the leg spring 18. The aneurism clip 1 is made from metal, for example from titanium. Furthermore, the aneurism clip 1 is in the form of a one-piece and, resp., single-material and, resp., integral component.

FIG. 2 shows the aneurism clip 1 of FIG. 1 in a slightly opened state directly before a change of pivot point. By applying the force F to the force-guiding path 23 the clip branches 2, 3 were deflected about the leg spring pivot point 19 so that the distal ends 4, 5 form a branch opening angle 9. The branch opening angle φ is located, in the configuration shown in FIG. 2, within a range between 3° and 10° and corresponds to the predetermined branch opening angle φ_{predetermined} according to the invention. Another deflection of the clip branches 2, 3 about the leg spring pivot point 19 is prevented by the fact that the pin 21 of the pin joint 20 now is arranged at the other, lower end in FIG. 1, of the slotted hole recess 22. A further deflection of the distal ends 4, 5 of the clip branches 2, 3 about the leg spring pivot point 19 is blocked in this way. When now, starting from the state shown in FIG. 2, a force F is continued to be applied to the spring portions 10, 11, the force F becomes large enough preferably directly after the blocking of the translational motion of the pin 21 so as to initially deflect and, resp., to bend the leaf springs 16, 17. A very small further deflection of the spring portions 10, 11 about the leg spring pivot point 19 preferably entails the deflection and, resp., bending of the leaf springs 16, 17. By the fact that the leaf springs 16, 17 are bent and by the fact that the force-guiding path 23 is arranged distally from the pin joint 20, now rotation of the distal ends 4, 5 of the clip branches 2, 3 together with the connecting portions 12, 13 about the pin 21 is enabled. Thus, in the configuration shown in FIG. 2 a change of the pivot point from A to B and, resp., from the leg spring pivot point 19 to the pin 21 takes place.

It becomes evident from this description that the configurations or structural designs and, resp., mechanical properties of the leaf springs 16, 17, of the leg spring 18 as well as of the slotted hole recess 22 have to be precisely adapted to one another. Especially the leaf springs 16, 17 shall be prevented from bending already during the translational motion of the pin 21 in the slotted hole recess 22. Moreover, the leaf springs 16, 17 are to be formed so that, at the beginning of the working area of the leaf springs 16, 17, a resulting force to be applied to the force-guiding path 23 is increasing with an increasing deflecting angle to a smaller extent than in the working area of the leg spring 18. According to the invention, a clear separation is thus provided between the working area of the leg spring 18 and the working area of the leaf springs 16, 17.

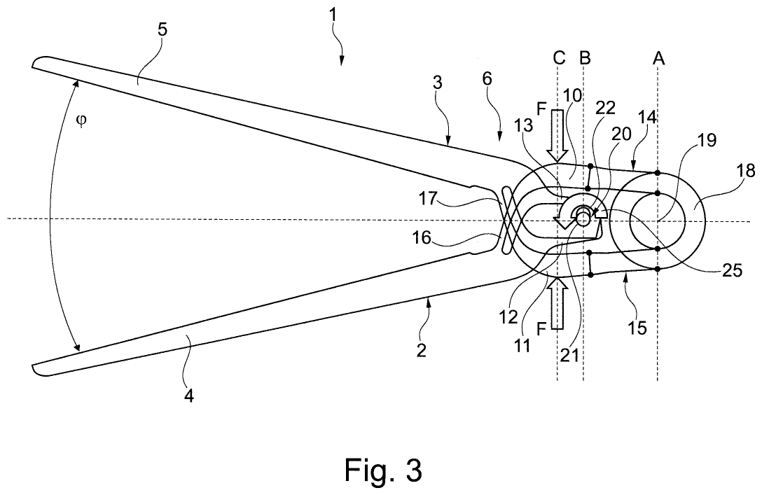

FIG. 3 shows the aneurism clip 1 of FIG. 1 and FIG. 2 in a widely opened state after a change of the pivot point from A to B. When starting from the state shown in FIG. 2 a force is continued to be applied to the force-guiding path 23 at C, the leaf springs 16, 17 will bend and the connecting portion 12 will rotate together with the distal end 4 about the pivot point at B formed by the pin 21 in the direction of rotation 25 indicated in FIG. 3. The connecting portion 13 and the distal end 5 rotate appropriately against the direction of rotation 25 indicated in FIG. 3. By the change of the pivot point from A to B a strongly converted rotation of the distal ends 4, 5 of the clip branches 2, 3 is obtained so that now a large branch opening angle φ is reached. The branch opening angle φ in FIG. 3 amounts to approx. 30° to 35°. When, starting from the configuration of FIG. 3, the distal ends 4, 5 of the clip branches 2, 3 are closed again, at first the leaf springs 16, 17 relax again and return to their non-bent state and the configuration of FIG. 2 is obtained again. Starting from the latter, upon closing in turn a translational motion of the pin 21 occurs in the slotted hole recess 22 and the distal ends 4, 5 again get to contact each other, wherein the closing force is applied to the clip branches 2, 3 merely by the leg spring 18. Thus, the leaf springs 16, 17 do not influence the closing force of the aneurism clips 1 in any way.

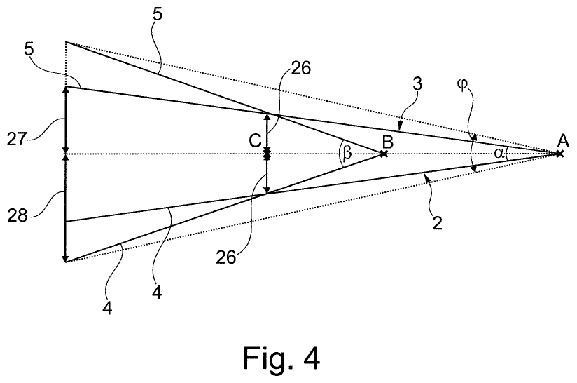

FIG. 4 illustrates a schematic view which illustrates the effects of the change of pivot point according to the invention on the opening width/the branch opening angle φ of the two clip branches 2, 3. Accordingly, a force which in turn results in a deflection 26 at C is applied to the clip branches 2, 3 at C. According to the invention, thus an angle α (opening angle at A) is enclosed between the two clip branches 2, 3. When the angle α reaches the afore-described branch opening angle φ_{predetermined}, a first branch opening width 27 is given and a rotation of the clip branches 2, 3 about the point A is blocked. For this, for example within a range of 0°≤φ≤10° a force F=k_{A}×α is required, wherein k_{A} denotes the spring constant of the (leg) spring at A. When further applying force at C, the pivot point is changed from A to B. Now the distal ends 4, 5 of the clip branches 2, 3 are further opening and at the new pivot point B enclose an angle β (opening angle at B) with a second branch opening width 28. For example, the change of pivot point occurs with φ=10° and for a range of φ>10° a force F=k_{A}×α+k_{B}×β is required, wherein k_{B} denotes the spring constant of the leaf or flexible spring(s) which are bent for further deflection. Here k_{B} is preferably smaller than k_{A}. A change of the pivot point from A to B, as provided according to the present invention, thus preferably results with a lower force application—as compared to a rotation of the clip branches 2, 3 merely about A in a larger obtainable branch opening width q at the distal ends 4, 5.

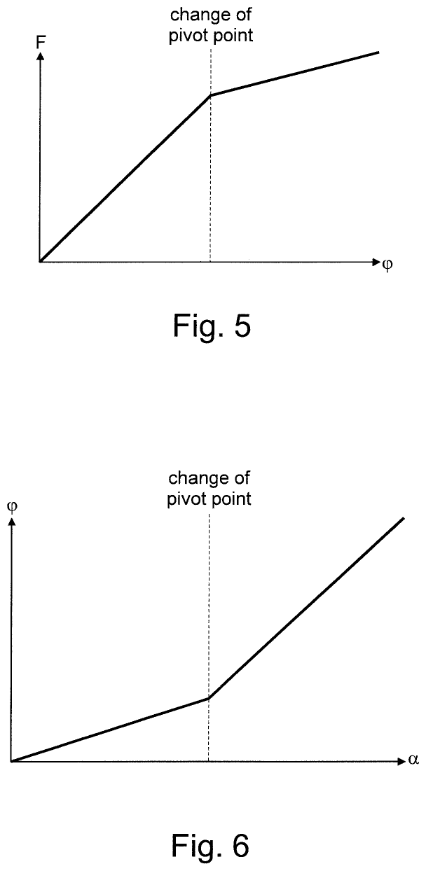

FIG. 5 shows a diagram which illustrates a relation according to the invention between a branch opening angle φ at the distal ends 4, 5 of the clip branches 2, 3 and a resulting force to be applied. In a working area of the leg spring 18 initially the force to be applied increases linearly and continuously with an increasing branch opening angle φ. The resulting force to be applied and the deflecting angle thus are proportional to each other. From the change of pivot point from A to B and thus with the beginning of the working area of the leaf springs 16, 17 the force to be applied linearly increases with an increasing branch opening angle φ to a smaller extent.

FIG. 6 shows a diagram which illustrates a relation according to the invention between a deflecting angle at the force-guiding path and, resp., an opening angle at A (angle α) and a resulting opening angle of the clip branches φ. Here initially in the working area of the leg spring 18 the resulting clip branch opening angle φ increases with an increasing deflecting angle α at the force-guiding path 23 to a small extent. Due to the change of pivot point from A to B in the working area of the leaf springs 16, 17 the resulting clip branch opening angle q linearly increases to a considerably greater extent due to the converted rotation.

1. A surgical clip comprising two clip branches, the clip branches comprising a first clip branch and a second clip branch, the clip branches being spring-elastically joined and pretensioned at proximal ends thereof via a first spring element which can be rotated about a spring pivot point formed by the first spring element, and which are held with distal free ends thereof in parallel against one another in a resting position of the surgical clip via a closing force of the first spring element, wherein the surgical clip has a conversion device which reduces a ratio of opening force, applied to a force-guiding path for opening the clip branches, to opening angle, from a predetermined opening angle of the distal free ends of the clip branches.

2. The surgical clip according to claim 1, wherein at least one of the clip branches has an additional pivot point which is arranged distally from the spring pivot point and proximally from the force-guiding path and about which the at least one of the clip branches can be rotated while overcoming a spring-elastic pretension of at least a second spring element.

3. The surgical clip according to claim 2, wherein each of the clip branches has an additional pivot point about which the clip branches can be rotated while overcoming a spring-elastic pretension of the second spring element and a third spring element.

4. The surgical clip according to claim 1, further comprising a spring travel limiting unit which limits a deflection and/or an opening angle of the clip branches about the spring pivot point formed by the first spring element.

5. The surgical clip according to claim 3, wherein the clip branches include each of a spring portion and a connecting portion at crossing portions where the clip branches are mutually crossing, wherein the spring portion of the first clip branch includes the second spring element and the spring portion of the second clip branch includes the third spring element, and wherein each of the spring portions is connected to the first spring element, the connecting portions of the clip branches being rotatably connected to each other such that the additional pivot points of the clip branches are in the form of a joint additional pivot point and each of the clip branches can be rotated about the joint additional pivot point via deflection and/or bending and/or overcoming of the spring-elastic pretension of the second and third spring elements.

6. The surgical clip according to claim 5, wherein the connecting portions of the clip branches are rotatably connected to each other via a pin joint, wherein the connecting portion of the first clip branch has a pin projecting in a direction of the second clip branch and the pin engages in a recess of the second clip branch and, in this way, a hinge-type rotatable connection is formed between the connecting portions of the clip branches, with the additional pivot point being formed by the pin.

7. The surgical clip according to claim 6, wherein the pin has a rotational degree of freedom and a translational degree of freedom, and a spring travel limiting unit is formed by limitation of translational movement of the pin.

8. The surgical clip according to claim 6, wherein the recess of the second clip branch is a slotted hole, wherein the pin is arranged, in a resting position of the surgical clip, at a first rounded side of the slotted hole, is translationally movable within the slotted hole by introduction of pressure for opening the surgical clip, and the spring travel limiting unit is formed by applying the pin to a second rounded side of the slotted hole.

9. The surgical clip according to claim 3, wherein the second and third spring elements are formed integrally and/or of one single material with the clip branches.

10. A method for opening the surgical clip of claim 1 comprising the steps of:
applying pressure to the clip branches on the force-guiding path formed between crossing portions and the first spring element and, in this way, deflecting the clip branches about the spring pivot point;
limiting the deflection about the spring pivot point by a spring travel limiting unit; and
further applying pressure to the clip branches and thus deflecting of at least one clip branch about an additionally provided pivot point which is arranged distally from the spring pivot point and proximally from the force-guiding path while overcoming a spring-elastic pretension of at least a second spring element.
